(19) United States
(12) Patent Application Publication
(10) Pub. No.: US 2004/0049201 A1
(43) Pub. Date: Mar. 11, 2004

(54) Implant comprising a grooved structure

(76) Inventor: Wolfgang Dinkelacker, Sindelfingen (DE)

Correspondence Address: KUDIRKA & JOBSE, LLP, ONE STATE STREET, SUITE 800, BOSTON, MA, 02109, US

(21) Appl. No.: 10/450,880

(22) PCT Filed: Aug. 3, 2002
(86) PCT No.: PCT/EP02/08679

(30) Foreign Application Priority Data
Aug. 8, 2001 (DE) 201 13 183.8
Jul. 24, 2002 (DE) 202 11 202.0

(51) Int. Cl. A61F 5/00

(52) U.S. Cl. 606/86

(57) ABSTRACT

The surface of a bone implant disposed with a plurality of grooves (20) along its longitudinal axis or at a sharp angle thereto which form an angle α radial the longitudinal axis (41) of the implant body (11) in at least one of multiple sections (16) and have different depths across their lengths. Said grooves may be configured in wedge or diamond shape or take the form of a curved wedge (231) or of elongated curved segments (251) of varying depths across their lengths and extending in axial, spiraling or crosswise-coiling manner relative the perimeter of the implant. Such a configured surface structure affords an improved deposition of bone tissue osteons. A titanium layer sputtered on the implant surface allows for osteocytes to collect in the troughs thereby created. The surface structure can be utilized preferably in the case of tooth implants but also in other bone implants including those having curved longitudinal axes.

### SCOPE OF THE INVENTION

The present invention relates to an implant insertable into the human body during an operation having an implant body, the surface of which has a plurality of groove-shaped recesses, as well as a method for its manufacture.

### STATE OF THE ART

The applicant's EP 1 013 236-A discloses a cylindrical or conical tooth implant, the surface of which has a plurality of groove-shaped recesses running along its longitudinal axis or at a sharp angle thereto, and which may also be arranged to run crosswise. In another embodiment of the implant, the surface of the implant body is divided along its longitudinal axis into numerous sections or steps separated from one another by radial bands and having a plurality of peripheral groove-shaped recesses. The dimensioning to the groove-shaped recesses is adapted to the dimensions of the osteons of the jawbone tissue which attach to said groove-shaped recesses. In manufacturing the tooth implant, a cylindrical, conical or stepped implant body is pre-formed with a smooth surface into which the groove-shaped recesses can then be created by means of a material removal process. A plurality of small, spatially and densely-distributed concave recesses are provided in the groove-shaped recesses to receive the osteocytes of the bone tissue surrounding the implant and hence serving to further improve the contact between the implant and the bone.

Such a surface structure allows bone tissue osteons to attach to the groove-shaped recesses and to grow into the implant. The dimensioning of the groove-shaped recesses being adapted to the dimensions of the osteons and their receiving of those osteons coming into contact with the implant favorably facilitates the growing in of the implant. As a result of this surface structure and due to the pressure acting upon the implant, compacta forms around the implant in the spongiosa region of the bone which affords a good absorption of the forces acting upon the implant as well as a stable and permanent seat for the implant.

### SUMMARY OF THE INVENTION

The present invention further enhances the described effect based upon a further improved adaptation of the surface structure of an implant of the type described above to the osteon profile and the behavior of the osteons during the healing phase.

In accordance with the invention, as defined by the claims, the groove-shaped recesses on the surface of the implant are inclined toward the longitudinal axis of the implant body. They form an angle α to the longitudinal axis and are of varying depths across their length. The surface structure which thus results is accommodating of bone tissue anatomy. It has been found that the osteons accumulating on the implant tend to align less toward the implant axis than they do transverse thereto. Such a depositing of osteons on the implant surface mainly ensues at an acute angle, which represents a spatial condition for the accumulating of a large number of osteons. The osteons can hereby grow into the groove-shaped recesses at an oblique angle from above or at an oblique angle from below. The canted profile to the groove-shaped recesses supports this form of accumulation, improves the contact between the bone tissue and the implant, and shortens the healing phase.

Further improvement is attained by curvature across the lengthwise extension of the grooves. According to one aspect of the invention, the grooves are configured such that in an implementation of the peripheral region, their profile exhibits a curved wedge shape or is in the shape of elongated curved segments.

According to an inventive method for manufacturing the grooved structure, a blank of the implant body is made in which grooves are be produced in at least one of its sections in a material removing process. Said grooves form an angle α radial the longitudinal axis of the implant body and their depths vary across their lengths. The material removing process preferably utilizes metal cutting and a synchronous feed of implant body and the cutting tool which can be varied from section to section. The synchronous feed is preferably selected such that the grooves will have a curvature to at least one of their sections. According to a further aspect of the invention, the synchronous feed is preferably selected such that the grooves will be narrow and shallow at both edges of a section and exhibit their maximum width and depth at their center.

In accordance with a further step of the inventive procedure, a metal layer is sputtered on the surface of the grooves, said layer preferably being a titanium layer.

### DESCRIPTION OF THE DRAWINGS

Various embodiments of the invention will be depicted in the following in conjunction with the drawings, which show:

FIG. 1: an embodiment of a tooth implant according to the present invention having a cylindrical implant body divided into various sections;

FIG. 2: a partial section along line 2-2′ from FIG. 1;

FIG. 3: a schematic representation of an implementation of a peripheral segment of one of the sections from FIG. 1;

FIG. 4: a partial section along line 4-4′ from FIG. 3;

FIG. 5: a schematic representation of an implementation of a peripheral segment comprising grooves exhibiting increasing groove depth toward the head of the implant;

FIG. 6: a section along line 6-6′ from FIG. 5;

FIGS. 7-8: schematic representations of implementations of a peripheral segment of the implant exhibiting grooves spiraling to the left or to the right;

FIGS. 9-10: sections along line 9-9′ from FIG. 7 and line 10-10′ from FIG. 8;

FIGS. 11-12: schematic representations of implementations of a peripheral segment of the implant exhibiting grooves spiraling crosswise to the left and right;

FIGS. 13-14: schematic representations of implementations of a peripheral segment of the implant having diamond-shaped grooves;

FIG. 15: a section along line 14-14′ from FIG. 13;

FIGS. 16-18: embodiments of the inventive implant having a stepped implant body;

FIGS. 19-20: embodiments of the inventive implant having a conical implant body;

FIGS. 21-22: embodiments of the inventive implant having an implant body comprising a combination of groove structures;

FIGS. 23-24: schematic representations of implementations of a peripheral segment of an inventive implant comprising curved grooves having a profile in the form of a curved wedge and spiraling to the left or to the right;

FIG. 25: a schematic representation of the implementation of a peripheral segment of an inventive implant comprising curved grooves having a profile configured as elongated curved segments;

FIG. 26: a schematic representation of the implementation of a peripheral segment of an implant having curved grooves of the type as depicted in FIG. 25 spiraling crosswise;

FIG. 27: an embodiment of the implant having a stepped implant body exhibiting the type of groove structure combination as depicted in FIGS. 25 and 26; and

FIG. 28: an embodiment of the inventive implant having a curved longitudinal axis as may be utilized, for example, in hip joint replacements.

### DETAILED DESCRIPTION OF THE EXEMPLARY EMBODIMENTS OF THE INVENTION AS DEPICTED IN THE DRAWINGS

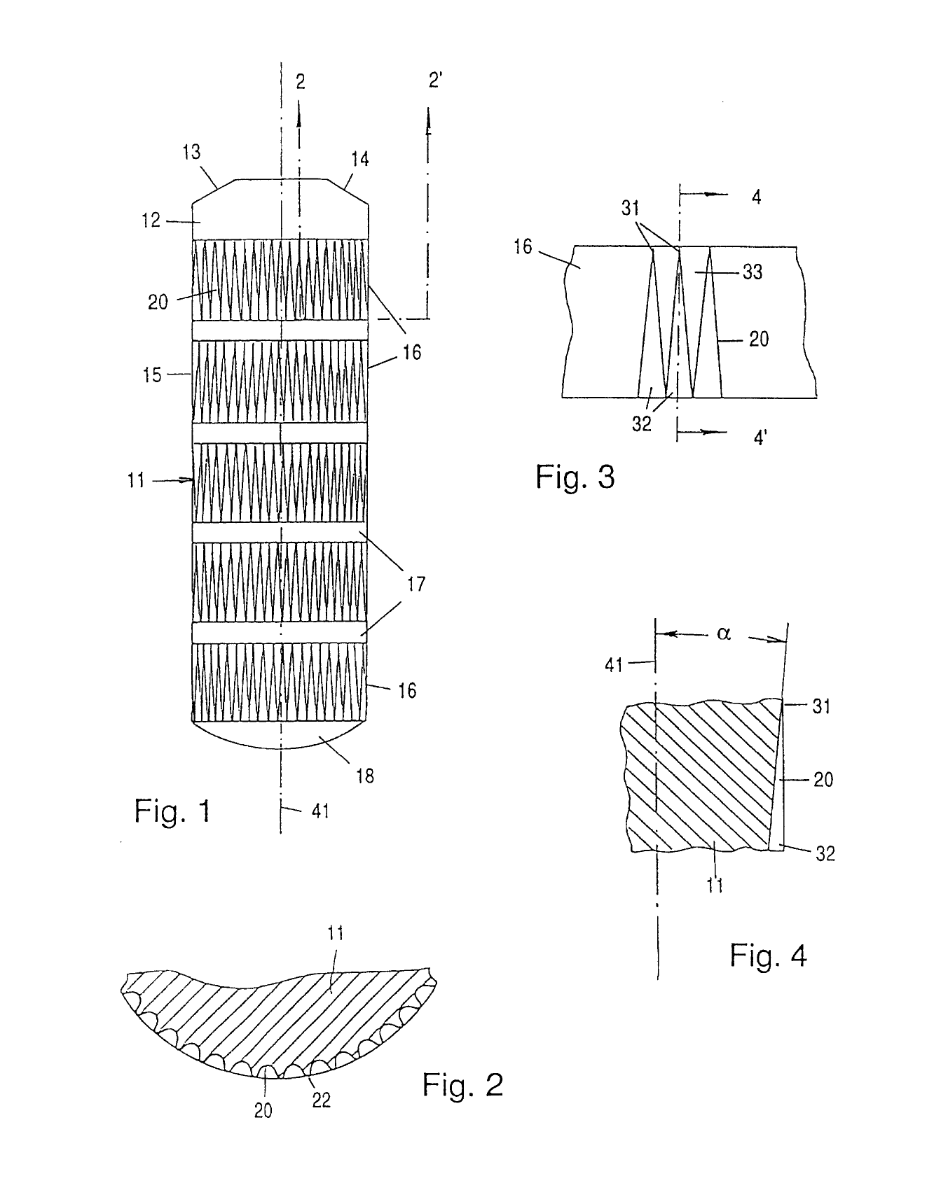

The tooth implant depicted in FIG. 1 comprises a cylindrical implant body 11 made from titanium, ceramic or other sufficiently hard enough material tolerated by the bone tissue of a human body. Implant body 11 has a head area 12 disposed with bevels 13, 14 on its bucal and lingual sides as disclosed in EP-A 0 868 889. Said head area 12 serves to receive supports (not shown) for a tooth crown. A body area 15 divided into sections 16 is adjoined to head area 12, bands 17 being disposed between said sections. The example depicted in FIG. 1 exhibits five sections 16 separated by four bands 17. The base of implant body 11 terminates in a rounded foot area 18.

The perimeters of sections 16 are disposed with densely-distributed groove-shaped recesses 20, referred to in the following simply as grooves, running parallel to the implant axis relative the perimeter of implant body 11. Grooves 20 preferably have a concave profile, the edges of which run to the perimeter of body area 15 and form a rounded comb 22 with each of both adjacent recesses at the area of greatest depth (FIG. 2 sectional representation). Grooves 20 have a width which is preferably in the range of between 0 and 300 micrometers and a depth which varies from between 0 micrometers and preferably up to 150 micrometers.

FIG. 3 shows a schematic representation of an implementation of one of sections 16 in which three grooves 20 are depicted. Grooves 20 form an angle α radial the longitudinal axis 41 of implant body 11 and are of varying depth across their length (FIG. 4). This results in grooves 20 exhibiting what approximates a wedge-shaped profile at the implementational level. The apexes 31 to the limiting lines are situated at the peripheral diameter at the upper edge of depicted section 16. The wedge shape exhibits its greatest width in area 32 at the lower edge of the section and, at the same time, this is where the grooves have their greatest depth, as is shown in FIG. 4. Very nearly wedge-shaped spans 33 running in the opposite direction are found between grooves 20 which widen through to apexes 31 of grooves 20, their height determining the perimeter of the section. It should be apparent that the limiting lines to grooves 20 in the actual implementation of the perimeter exhibit curvature at the imaging plane, which is not shown in FIGS. 3 and 4 for reasons of simplifying the representation.

Angle α is defined by the maximum depth of the grooves and the axial length of sections 16. With a maximum groove depth of 150 micrometers and a sectional length of roughly 2 mm, α has a value in the proximity of 4.5°.

The implant surface structure as described accommodates the anatomy of the jawbone tissue. During the implant healing phase, osteons of the bone tissue surrounding the implant can collect in grooves 20. It has been found that the osteons depositing on the implant tend to align less with the implant axis than they do transverse thereto. Such a depositing of osteons on the implant surface mainly ensues at an acute angle, which represents a spatial condition for the accumulating of a large number of osteons. The osteons can hereby grow into grooves 20 at an oblique angle from above or at an oblique angle from below. The canted profile to grooves 20 supports this form of accumulation, fosters a close contact between the jawbone tissue and the implant, and shortens the time period needed for growing in.

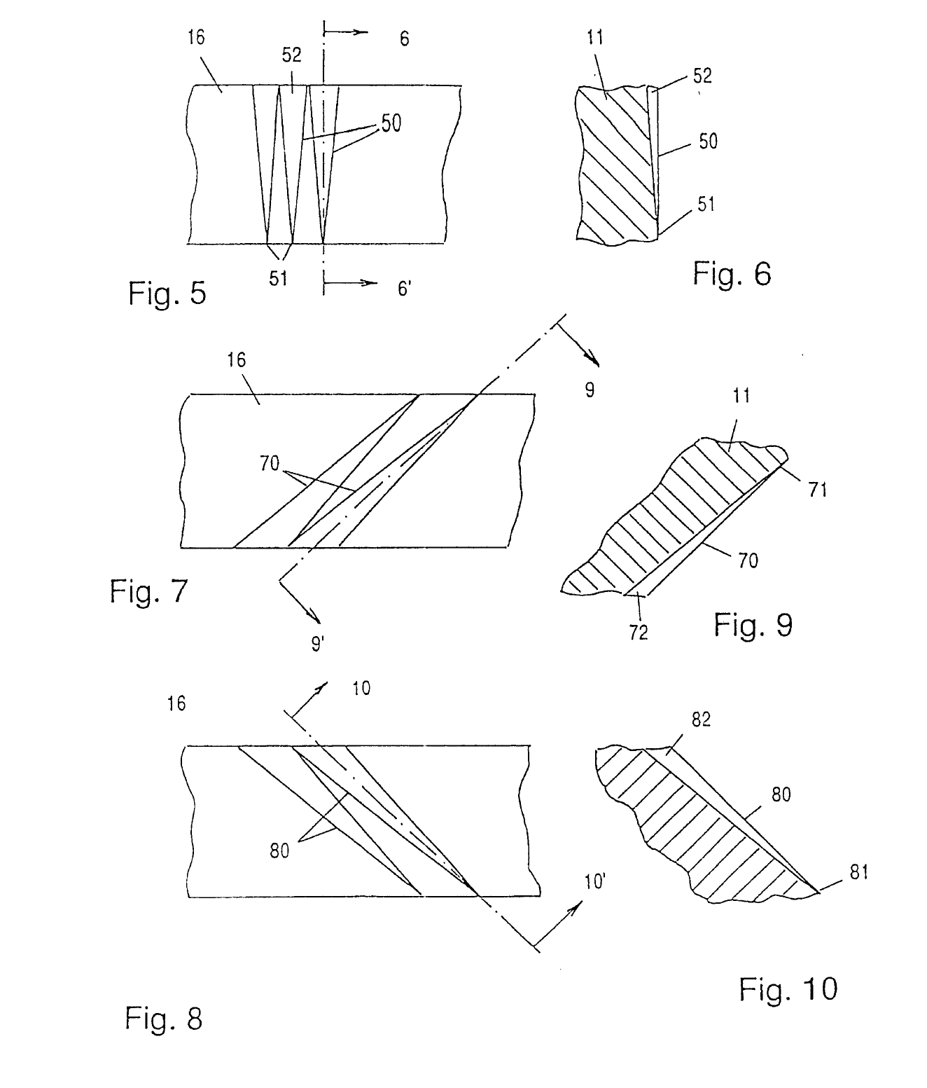

Deviating from the form depicted in FIGS. 1-4, the grooves may also be arranged differently or have a different shape. FIGS. 5 and 6 show a groove profile to grooves 50 which has been rotated 1800 compared to the groove profile depicted in FIGS. 3 and 4; i.e., apexes 51 of grooves 50 are positioned at the lower edge of depicted section 16 and the greatest groove depth is at area 52 at the upper edge of said section 16, as shown in the FIG. 6 sectional representation.

FIGS. 7 and 8 show an implementation to sections 16 of implant body 11 in which the grooves have a spiraling profile relative the perimeter of implant body 11. FIG. 7 shows a progression to grooves 70 which winds to the right and which additionally extends in wedge-shaped form from upper edge 71 of section 16 to the lower edge of said section, area 72, having the greatest depth to grooves 70 (FIG. 9). FIG. 8 shows a progression to grooves 80 which winds to the left, extending from lower edge 81 of section 16 to the upper edge of said section which is, in this case, area 82 showing the greatest depth to grooves 80, as shown in the FIG. 10 sectional representation.

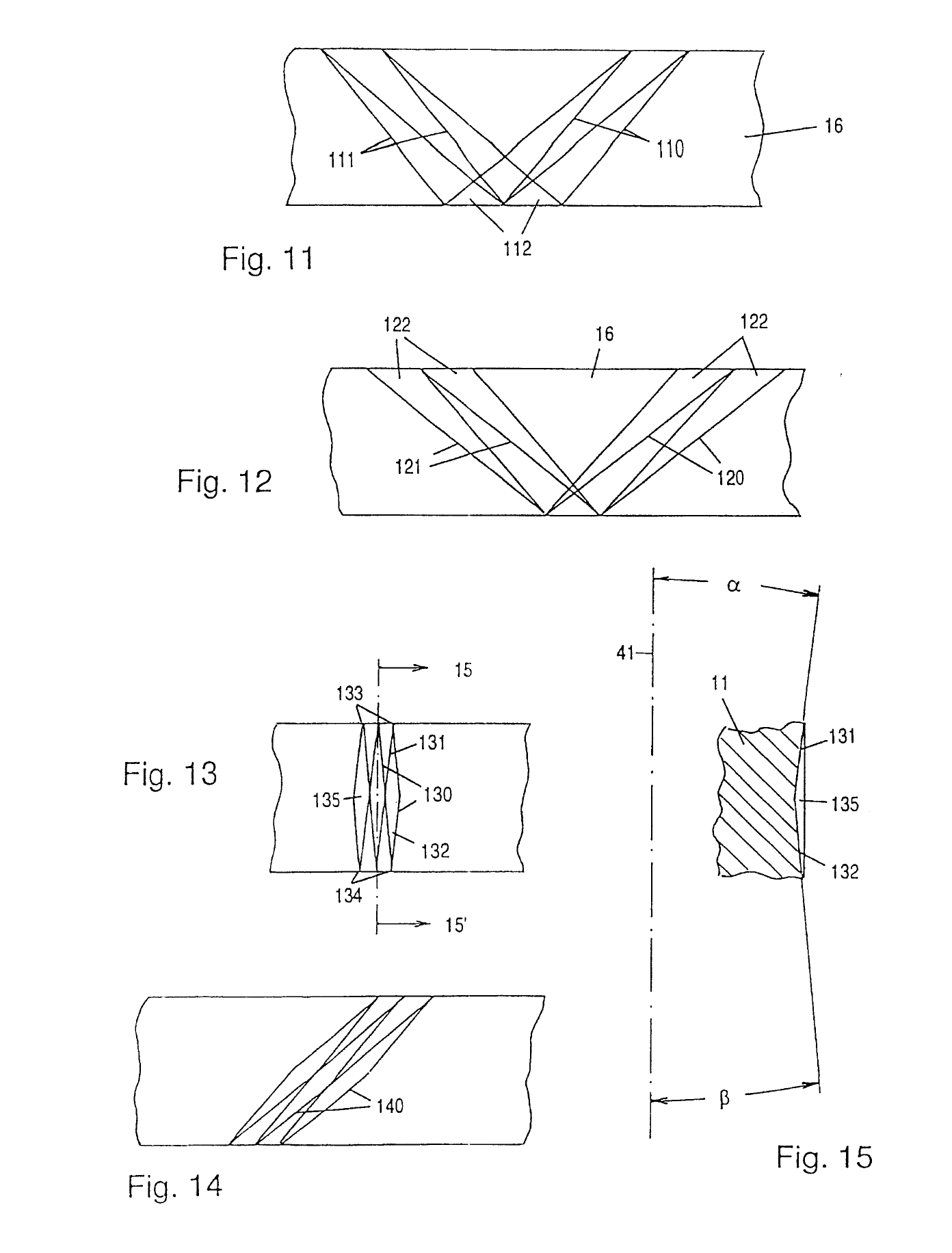

FIGS. 11 and 12 show implementations of a crosswise progression to the grooves. The embodiment according to FIG. 11 provides for first grooves 110 extending in a first direction winding to the left at an acute lead angle and second grooves 111 extending at an acute lead angle in a second direction winding to the right and intersecting with first grooves 110. Grooves 110 and 111 have a wedge-shaped profile from the upper edge of section 112 to the lower edge of said section, area 112, being the region of greatest depth for grooves 110 and 111. Such a groove progression produces the cut spans 33 as depicted in FIG. 3 resulting in a plurality of knob-shaped protrusions, their height extending to, respectively defining, the perimeter of sections 16.

In the embodiment according to FIG. 12, grooves 120 and 121 exhibit a crosswise progression like grooves 110 and 111 from FIG. 11, although grooves 120 and 121 are arranged here such that the wedge-shaped progression runs from the lower edge of section 122 to the upper edge of said section, the area of greatest depth to grooves 120 and 121.

In the embodiment according to FIGS. 13-15, the grooves exhibit what approaches a diamond-shaped profile in the implementation of the perimeter which results in a pair of wedge-shaped grooves set in arrangement against one another. FIG. 13 shows the implementation of a section 16 of implant body 11 having grooves 130, the limiting lines of which extend in approximate diamond shape. Grooves 130 are arranged densely adjacent one another and parallel to the implant axis relative the perimeter of the implant body. Each of grooves 130 is comprised of two wedge-shaped groove segments 131, 132, wherein the profile to the groove of the one groove segment 131 forms an angle α to the implant's longitudinal axis 41 and the profile of the groove to the other groove segment 132 forms an angle β to the implant's longitudinal axis 41 (FIG. 15). The two outermost apexes 133 and 134 of each of grooves 130 are thus, in this embodiment, situated on the peripheral line of section 16, and the area 135 of maximum depth of each one of grooves 130 is at its center where the two groove segments 131 and 132 meet.

FIG. 14 shows the implementation of an implant body section 141 comprising grooves 140 which likewise have a diamond-shaped configuration to their limiting lines. Yet grooves 140 extend here in coiling fashion relative the perimeter of the implant body as described above with reference to FIGS. 8 and 9. In all other respects, the configuration of grooves 140 corresponds to that of grooves 130 in FIG. 13.

The groove structures shown in FIGS. 5-15 provide favorable conditions for osteons to accumulate during the healing phase and additionally secure against axial shifting/rotation of the implant once ingrown. According to need, various different groove structures can also be advantageously combined within one implant, as described with reference to FIGS. 21 and 22, for example.

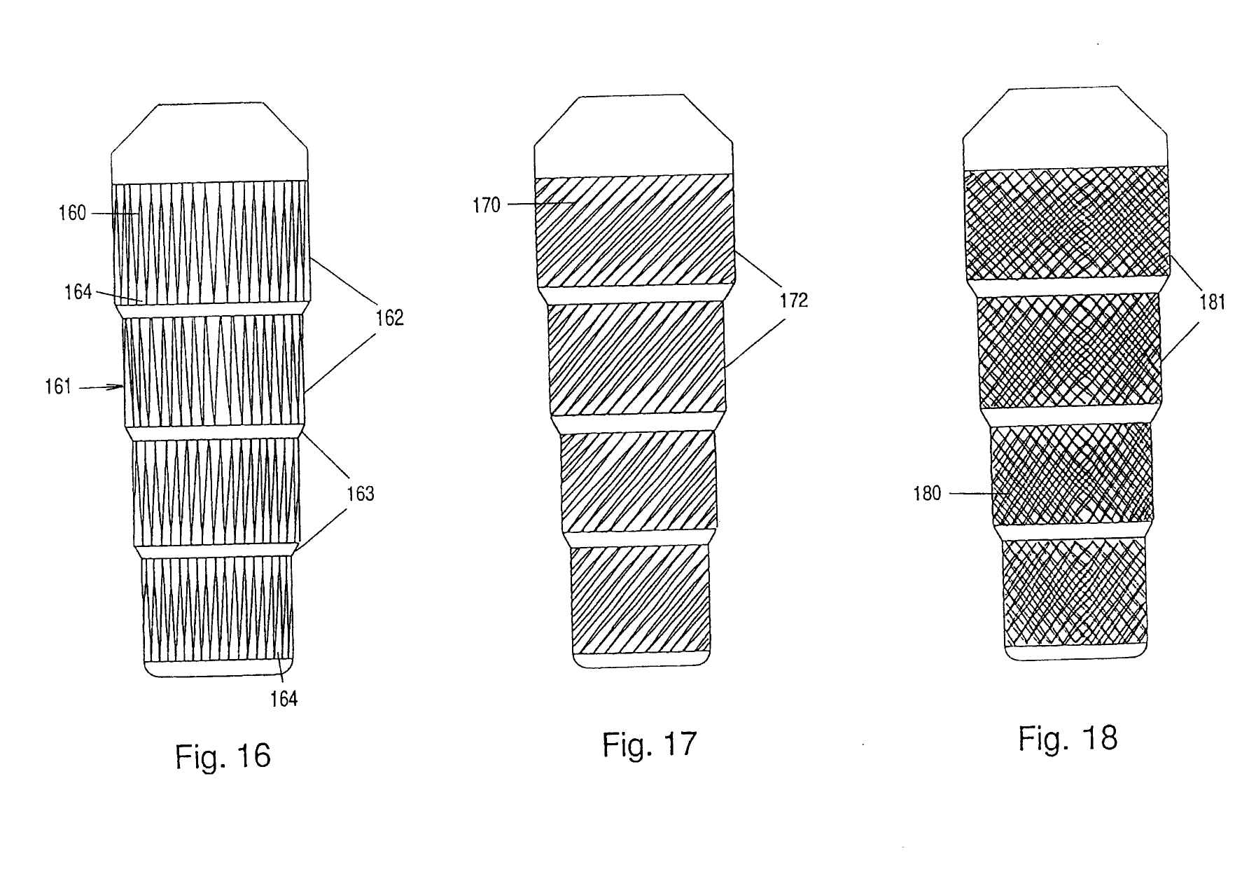

Drawing upon FIGS. 16-22, the following will detail different embodiments of tooth implants which make use of such groove structures as described above. The tooth implants as depicted are preferably made from titanium.

FIGS. 16 through 18 show embodiments of tooth implants in which the implant body has gradations in diameter. A plurality of such stepped diameters may be provided, whereby the height of a gradation measured at the diameter is preferably of a magnitude ranging between 20 and 300 micrometers. The embodiment depicted in FIG. 16 comprises an implant body 161 having four steps 162. Conical transition areas 163, also referred to herein as bands, which are narrow in relation to the length of the steps, are arranged between said steps. The perimeter of each one of steps 162 is disposed with a groove structure of the type depicted for grooves 20 in FIGS. 3-15. Grooves 160 are provided at the periphery of each one of steps 162, extending along the longitudinal axis of implant body 11 in relation to the perimeter, said grooves being the type of grooves 20 as described in conjunction with FIGS. 3 and 4. In area 164 of greatest depth, grooves 160 all have the same width, which results in a differing number of grooves 160 within each step 162. In the case of implants in accordance with FIG. 17, grooves 170 which wind to the right are arranged at the perimeter of steps 172, said grooves being the type of grooves 70 as described in conjunction with FIGS. 7 and 9. In all other respects, the implant according to FIG. 17 corresponds to the FIG. 16 implant. The implant according to FIG. 18 differs from the implant according to FIG. 17 in that the peripheral area of each one of steps 181 is provided with cross-wise grooves 180 in the manner of grooves 111 as described in conjunction with FIGS. 11 and 12. Grooves 160, 170 and 180 are only depicted schematically in FIGS. 16-18.

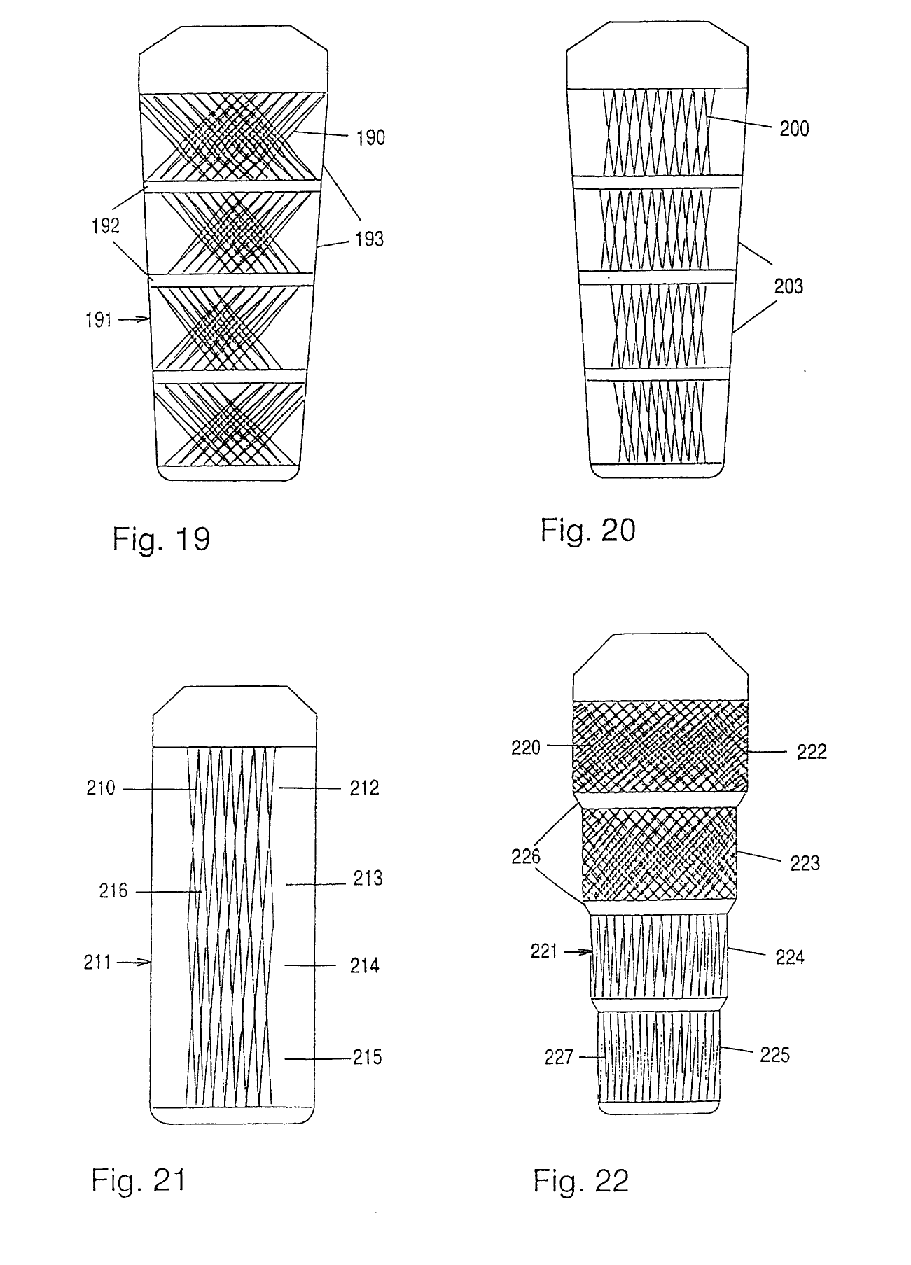

FIGS. 19 and 20 show embodiments of tooth implants having a conical implant body. In the embodiment according to FIG. 19, the conical implant body 191 is divided into sections 193 by bands 192 running along the perimeter. Crosswise grooves 190 are disposed at the periphery of sections 195, said grooves being the type of grooves 111 as described in conjunction with FIGS. 11 and 12. FIG. 20 shows an embodiment in which the sections comprise diamond-shaped grooves 200 in the manner of the grooves as described in conjunction with FIGS. 13-15. In all other respects, the implant according to FIG. 20 corresponds to the implant depicted in FIG. 19. Grooves 190 and 200 are only partially shown in FIGS. 19 and 20; and in the case of these embodiments as well, the groove structure as respectively described extends across the entire perimeter of sections 193 and 203.

FIGS. 21 and 22 show tooth implants in which each different section segment will exhibit different groove structures. FIG. 21 shows an implant having a cylindrical implant body 211 with a series of grooves 210 arranged at its periphery such that the implant body is divided into four sections 212, 213, 214, 215. The grooves of the uppermost section 212, situated closest to the implant head, comprise wedge-shaped grooves 210 at its periphery of the type of grooves 20 as described in conjunction with FIGS. 3 and 4. Section 213 which follows in the direction of the implant foot, has peripheral grooves 216 of reversed wedge configuration of the type of grooves 50 as described in conjunction with FIGS. 5 and 6. Section 214 adjoins thereto, having again the same groove configuration as section 212, and is then followed by section 215 having grooves of reversed wedge shape. Sections 212-216 merge together seamlessly and are only marked by the change in groove structure. Also in FIG. 21, only a portion of the grooves are shown for purposes of representation.

FIG. 22 depicts an embodiment of a tooth implant in accordance with the type of stepped implant as described in conjunction with FIGS. 16-18. In this embodiment, implant body 221 comprises groups of sections wherein the groove structure of one group differs from that of the other groups. Implant body 221 is divided into four steps 222-225, connected together by means of conical transition areas 226. The two uppermost steps 222 and 223 have crosswise grooves 210 at their periphery of the type of grooves as described in conjunction with FIGS. 11/12. Sections 224-225 which follow in the direction of the implant foot, have grooves 227 at their periphery of a wedge shape which runs the longitudinal direction of the implant in relation to the perimeter of implant body 221, said grooves being of the type of grooves 20 as described in conjunction with FIGS. 3 and 4.

By making use of a surface structure having varying steps within the same implant, the varying bone thicknesses surrounding the length of the implant can be taken into account. This allows, for example in the case of an implant according to FIG. 22, the two uppermost steps 222 and 223 with their crosswise-running grooves to provide a good anchoring for the implant within the compacta and the adjacent area, while the surfaces of the subsequent steps can be adapted to the bone tissue increasing in porousness further below. The combination of different surface structures across the length of the implant body also supports the objective of securing the implant against axial shifting/rotation during the healing and healed phases, and provides for a better conducting of pressure to the bone.

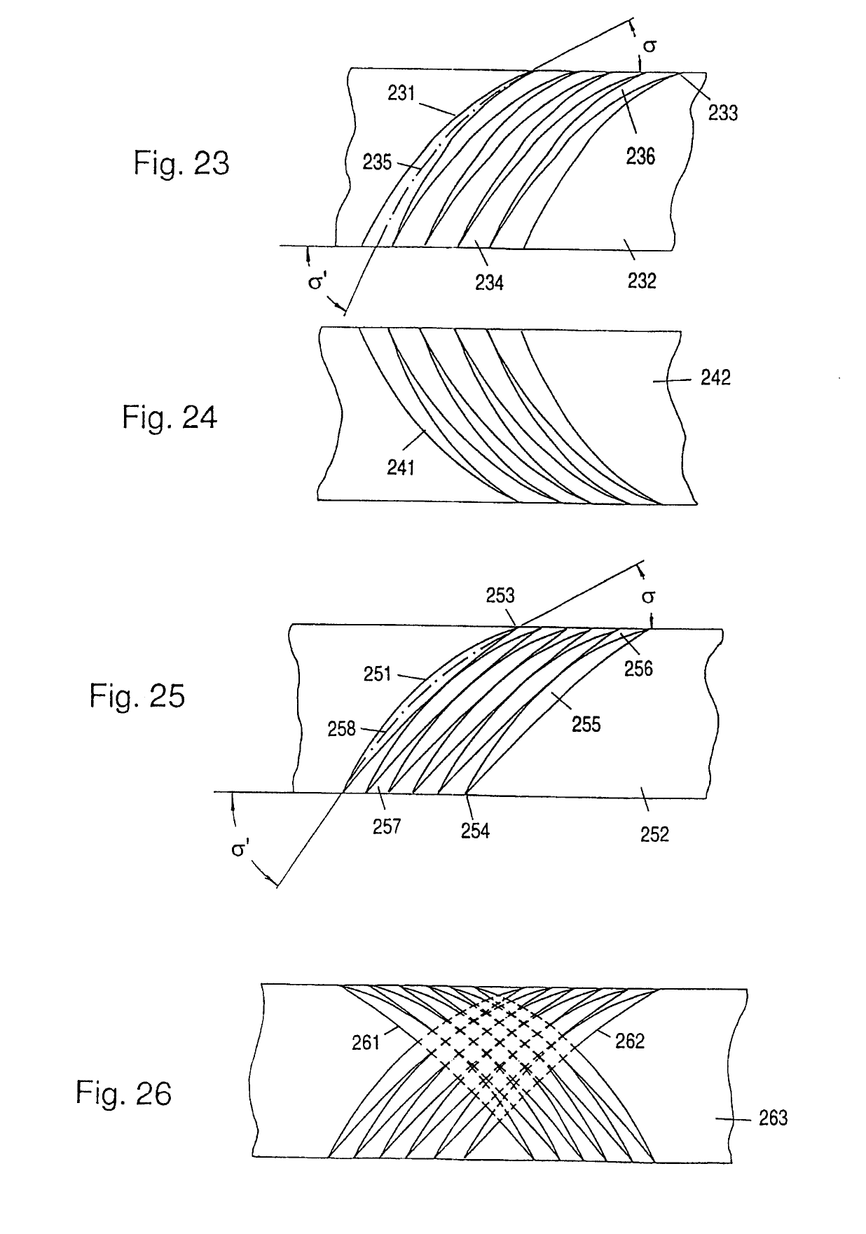

Examples of other embodiments of the present invention are depicted in FIGS. 23-28. These embodiments exhibit curved wedge-shaped grooves or grooves in the form of elongated segments of coiling or crosswise-coiling progression.

FIG. 23 shows the implementation of the perimeter of an implant section 232 having right-spiraling grooves 231 with a profile in the form of a curved or bending wedge. The degree of curvature to the wedge exceeds the above-cited curvature to the limiting lines of linearly straight grooves 20 in the actual implementation of the perimeter at the imaging plane and which are not shown in FIGS. 3-14 for reasons of simplifying the representation. The progression of depth to grooves 231 corresponds to the progression of depth as depicted for grooves 20 and 70 in FIGS. 4 and 9. Apex 233 of the curved wedge is situated at the diameter of the perimeter at the upper edge of depicted section 232. Area 234 is situated at the lower edge of section 232 where the curved wedge exhibits its greatest width and, at the same time, its greatest depth. The correspondingly inverted curved wedge-shaped spans 236 are situated between the densely-adjacent and adjoining grooves 231, widening to the apexes 233 of grooves 231 and with their height defining the perimeter of section 232. At area 233 of least groove depth, longitudinal axis 235 of grooves 231 forms an angle σ to the upper boundary of section 232, while longitudinal axis 235 forms an angle σ′ to the lower boundary of section 232 at area 234 of greatest groove depth, whereby σ<σ′.

FIG. 24 shows the implementation of the perimeter of an implant section 242 comprising left-coiling grooves 241 having a profile in the form of a curved or bending wedge as do grooves 231. In all other respects, the configuration and arrangement of grooves 241 corresponds to that of grooves 231 in FIG. 23.

FIG. 25 shows the implementation of a section 252 of an implant body comprising grooves 251 with a profile configured as an elongated curved segment in a shape similar to a banana. Grooves 251 are arranged densely adjacent one another and exhibit a coiled profile with respect to the implant body periphery, as described above, for example with reference to FIG. 14. Each of the grooves 251 can be considered as consisting of two seamlessly merging grooves 231, 241. As the progression of depth is concerned, that as realized for these grooves applies. The two outer apexes 253 and 254 of each one of grooves 251 is situated at the peripheral line of section 252, and the area of maximum depth of each one of grooves 251 may be in the proximity of the center of its longitudinal extension, as depicted in FIG. 25 with respect to area 255. The area of maximum depth to each one of grooves 251 may also be situated away from the groove center, for example in the first or last fourth or in the first or last third of the longitudinal extension to the grooves 251.

Correspondingly curved wedge-shaped spans 256 and 257 lie between adjacent grooves 251, widening in extension to apexes 253, 254 of grooves 251, their height defining the perimeter of section 252. Longitudinal axes 258 of grooves 251 form an angle σ to the upper boundary of section 252 at area 253 and an angle σ′ to the lower boundary of section 252 at area 254, whereby σ<σ′.

FIG. 26 shows the implementation of a section 263 of an implant body comprising grooves 261, 262 having a profile configured in the shape of an elongated banana-like curved segment of the type as grooves 251 in FIG. 25. Grooves 261, 262 extend crosswise to one another, as is described with respect to grooves 111 and 121 in conjunction with FIGS. 11 and 12. In the configuration according to FIG. 26, grooves 261 coil to the left and intersect with grooves 262 progressing in coiled fashion to the right. The depth progression to grooves 261 and 262 corresponds to the depth progression of curved grooves 251 as described in conjunction with FIG. 25. Due to this groove profile, spans are cut between adjacent grooves 261, 262 of the type as spans 256 and 257 so that a plurality of knob-like protrusions result, their height extending to the perimeter of section 263, defining same respectively.

The groove structures represented in FIGS. 23-26 offer an additional advantage in that the horizontally-aligned osteons of the compacta and the vertically-aligned osteons of the spongiosa will merge together.

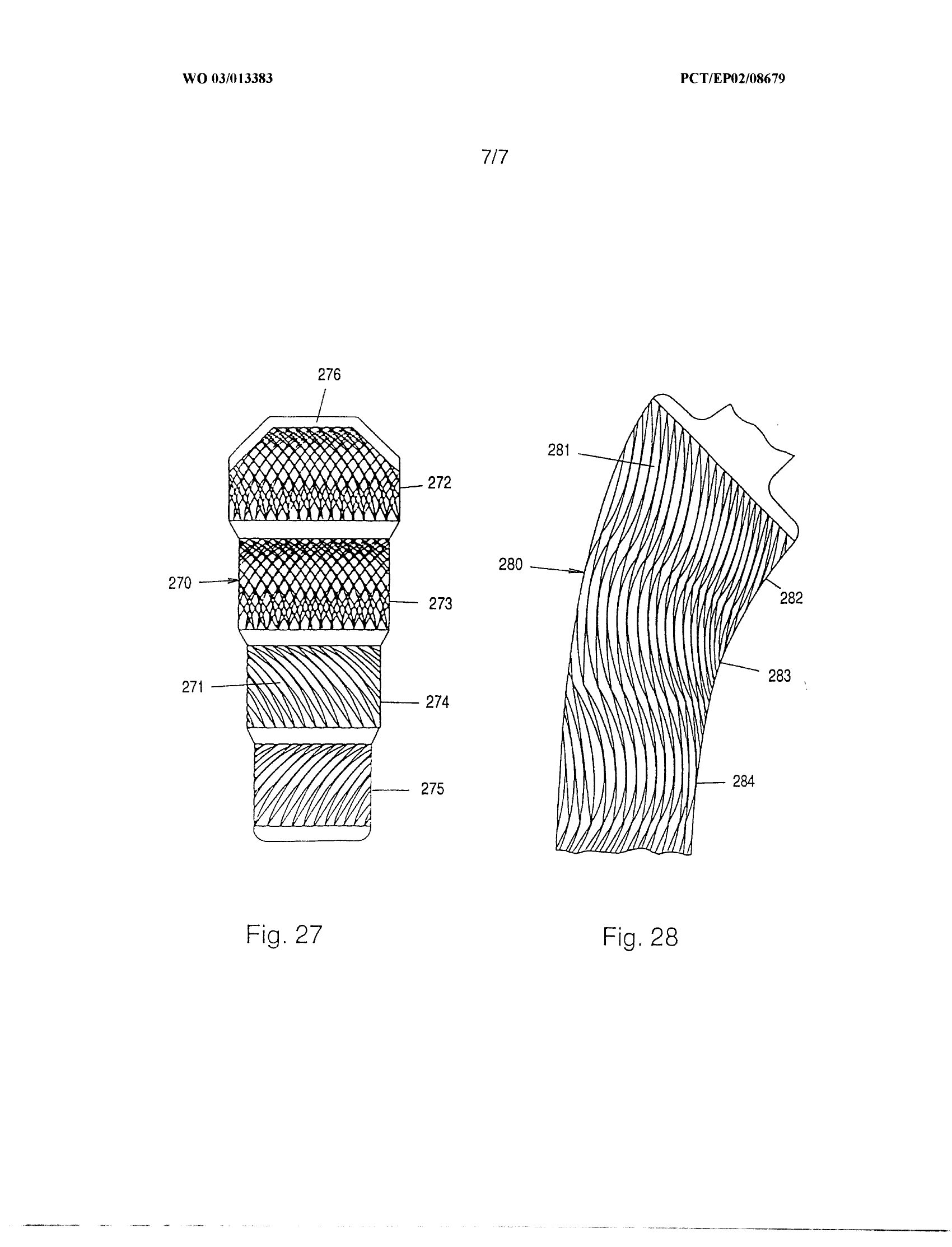

The groove structures according to FIGS. 23-26 can be combined within one implant subject to need. FIG. 27 shows an example of this, making use of a tooth implant 270 of the type as described above in connection with an embodiment of an implant configured in accordance with FIGS. 16-18 and 22 in which the implant body is configured as a stepped cone. Implant 270 comprises four sections 272-275, the upper sections 272, 273 of same having crosswise-coiling grooves of the type of grooves 261 and 262 of FIG. 26. Grooves 271 of the uppermost section 272 extend here in the head region of implant 270 up to a narrow edge region 276 which runs virtually parallel to the upper edge of the implant. Sections 274, 275 have grooves 271 winding to the left and the right, said grooves being of the type as depicted in FIG. 25.

In addition, groove structures as in accordance with FIGS. 23-26 can be used in cylindrical tooth implants of the implant type as depicted in FIGS. 1-21 as well as in conical tooth implants of the implant type as depicted in FIGS. 19 and 20.

The invention can be used in the case of implants having an implant body which is not rotationally symmetrical or which has a curved longitudinal axis, as can be the case, for example, with hip joint implants. FIG. 28 shows a segment 281 of such an implant having a longitudinal axis extending in curved fashion with sections 282, 283 and 284 arranged thereupon, each being of wedge-shaped cross-section and joining seamlessly with one another. Sections 282-284 have grooves 281 of the type of grooves 251 from FIG. 25 which wind to the left in head section 282, then wind to the right in section 283 which follows, and then again wind to the left in the following section 284. Segment 281 as depicted may exhibit a non-rounded cross-section in the area of sections 282-284, the grooves adapted accordingly as far as length, inclination and curvature.

The surface of the inventive implant is provided with a metal layer by means of sputtering which gives the surface the necessary roughness for fostering the depositing of osteons. The metal layer is preferably of titanium. The titanium layer will cover, on the one hand, the surface of the grooves of the configuration described in conjunction with FIGS. 2-15 or 23-26 and, on the other hand, also the spaces between the grooves, which also includes the surfaces of spans 33, 236, 256 and 257 in implants of the type as described in conjunction with FIGS. 3, 23 and 25, as well as the radial bands disposed between the sections as seen in the embodiments depicted in FIGS. 3, 16-20, 22 and 27. A surface given this type of treatment has the additional advantage that the osteocytes of the osteons can accumulate not only in the area of the grooves but also externally of same in the troughs of the metal layer created by the sputtering process.

A preferred method of manufacturing implants according to the present invention consists of manufacturing a blank of the implant body in conventional manner as a cylinder, or in conical or stepped conical form, or as an implant body having a curved longitudinal axis, in each case with a smooth surface. The blank implant body then undergoes a material removing process in which grooves of the types as depicted in FIGS. 2-15 or 23-26 are discretionarily created in at least one segment of the sections. The material removal process preferably consists of a metal cutting process with which the profile, form and dimensions to the grooves are created by synchronous feed of the implant body and the cutting tool. In this manner, the synchronous feed can be selected so as to produce wedge-shaped grooves which form a first angle α radial the longitudinal axis of the implant body over one segment of a section and form a second angle β radial the longitudinal axis of the implant body over another segment of the same section. The synchronous feed can furthermore be selected so as to produce grooves of diamond-like or curved configuration or grooves which take the form of elongated banana-like segments and extend in axial, spiraling or crosswise-coiling manner relative the perimeter of the implant. When producing the grooves in sequential implant sections, the synchronous feed can be changed from one section to the next in order to create a combination of sections having differing groove structures on the same implant. In so doing, the groove structure can be extended at the head area of implant 270 to a narrow edge region 276 which extends in virtually parallel manner to the upper edge of the implant.

In a further step, a metal layer is applied to the surface of the inventive implant by means of which the surface acquires the necessary roughness to foster the depositing of osteons. This ensues by metal being sputtered onto the surface of the implant, said metal preferably being titanium. The titanium layer coats the inventive implant's groove surfaces as well as the spaces between the grooves including spans 33, 236, 256 and 257 in the case of implants of the type as described in conjunction with FIGS. 3, 23 and 25, the radial bands between the sections in implants of the type as described in conjunction with FIGS. 3, 16-20, 22 and 27, and the head region 13, 276.

While the invention has been depicted and described on the basis of preferential embodiments, additional further variations and other embodiments of the invention may also be realized without resulting in any departure from the scope of the invention as defined by the claims.

1. A bone implant, especially a tooth implant, comprising an implant body (11) having at its periphery a plurality of grooves (20) arranged along the implant body longitudinal axis or at an acute angle thereto, the dimensioning of same corresponding to the dimensions of the osteons of the bone tissue which collect on the groove-shaped recesses, and the surface of which is divided into a number of sections (16) arranged along its longitudinal axis, characterized in that grooves (20, 50, 70, 110, 231, 255) form an angle α radial the longitudinal axis of implant body (11) in at least one of said sections (16) and are of varying depths across their length.

2. The implant according to claim 1, characterized in that grooves (20) have their maximum depth at one of the edge regions of section (15) and run to the perimeter of the opposite edge region of said section.

3. The implant according to claim 1, characterized in that grooves (20) exhibit an approximate wedge-shaped profile at the implemented perimeter along the longitudinal axis of the implant (FIG. 3).

4. The implant according to claim 3, characterized in that said approximate wedge-shaped grooves (20) extend in coiled fashion at the perimeter.

5. The implant according to claim 1, characterized in that said grooves (130) form a first angle α radial the longitudinal axis (41) of implant body (11) across a portion of one of sections (16) and form a second angle β radial the longitudinal axis (41) of said implant body (11) across another portion of the same section (16).

6. The implant according to claim 5, characterized in that grooves (130) exhibit an approximate diamond-shaped profile at the implemented perimeter.

7. The implant according to claim 5, characterized in that said approximate diamond-shaped grooves (140) extend in coiled fashion at the perimeter.

8. The implant according to claim 1, characterized in that the depth of the grooves increases from the upper edge of section (16) to the implant foot (18).

9. The implant according to claim 1, characterized in that the depth of the grooves increases from the lower edge of section (16) to the implant head (12).

10. The implant according to claim 1, characterized in that a section (213) with increasing depth to the implant head follows a section (212) with increasing depth to the implant foot.

11. The implant according to claim 1, characterized in that grooves (231, 241) have a peripheral curvature in at least one of sections (232, 242).

12. The implant according to claim 11, characterized in that the angle of curvature (σ) to grooves (231) at one edge of said section (232) is smaller than the angle of curvature (σ′) to the grooves (231) at the other edge of said section.

13. The implant according to claim 1, characterized in that grooves (231, 241) have a continuously changing width and depth in at least one of said sections (232, 242).

14. The implant according to claim 13, characterized in that said grooves (231, 241) are narrow and shallow at one edge of said section (232, 242) and have their maximum width and depth at the other edge of said section.

15. The implant according to claim 13, characterized in that said grooves (251) are narrow and shallow at both edges of said section and have their maximum width and depth therebetween.

16. The implant according to claim 15, characterized in that the profile to grooves (251) at the implemented perimeter takes the form of elongated curved segments.

17. The implant according to claim 15, characterized in that grooves (251) have their maximum width and depth in an area (255) in close proximity to the middle of their longitudinal extension.

18. The implant according to claim 15, characterized in that grooves (251) have their maximum width and depth in an area at one-third of their longitudinal extension relative one of the two edges.

19. The implant according to one of claims 1-18, characterized in that said grooves (281) are disposed at the perimeter of at least one or more sections (282, 283 or 284) arranged along a curved longitudinal axis of the implant body (280) and have a wedge-shaped profile.

20. The implant according to claim 19, characterized in that grooves (281) at the perimeter of said wedge-shaped sections (282, 283 or 284) have differing lengths.

21. The implant according to at least one of claims 1-20, characterized in that first grooves (110, 120, 261) oriented in a first winding direction are provided on the perimeter of at least one of sections (16) and second grooves (111, 121, 262) oriented to a second winding direction are provided on the perimeter of the same section (16) and intersect with said first grooves (110, 120, 261), and that said first and said second grooves form an angle radial the longitudinal axis of implant body (11) and have varying widths and depths across their lengths.

22. The implant according to at least one of claims 1-21, characterized in that radial bands (17, 163, 192, 226) are arranged between the sections into which run the shallow ends of grooves (20, 160, 190).

23. The implant according to one of claims 1-22, characterized in that the groove-shaped recesses (20) are arranged densely adjacent at their areas (32, 255) of greatest depth and exhibit a concave profile, the edges of which give way to spans (33, 256, 257) or knob-shaped protrusions at the perimeter of implant body (11).

24. The implant according to claim 1, characterized in that implant body (191) is of conical shape and grooves (192, 200, 231, 251) are disposed in sections (193) along the cone which approximate a wedge or diamond shape or have a curved wedge shape or the form of elongated curved segments (251).

25. The implant according to claim 1, characterized in that sections (162) have differing diameters decreasing toward the implant foot to form a stepped conical shape and that grooves are provided in said sections (193,272-275) along the cone which are of approximate wedge or diamond shape or which have a curved wedge shape or which are in the form of elongated curved segments (251).

26. The implant according to at least one of claims 1-25, characterized in that a combination of different groove forms is arranged on the perimeter of the implant body (211, 221, 276).

27. The implant according to claim 26, characterized in that said implant body (221, 270) is disposed with groups of sections (222, 223 and 224, 225, 272-275) and that the grooves (210) of one of said groups (222, 223, 272, 273) differ in form from the grooves (227, 271) of said other groups (224, 225, 273, 274).

28. The implant according to claim 26, characterized in that the grooves (210) of one group (222, 223, 272, 273) spiral crosswise and the grooves (227, 271) of another group (224, 225, 274 275) run along the implant longitudinal axis or in a single coiled direction.

29. The implant according to claim 1, characterized in that grooves (271) of the uppermost section (272) extend in the head area to an edge region (276) which runs virtually parallel to the head area boundary of the implant.

30. The implant according to at least one of claims 1-29, characterized in that grooves (20, 231, 251) have a maximum depth measured at the perimeter in the range of 150 micrometers.

31. The implant according to at least one of claims 1-30, characterized in that grooves (20, 231, 251) have a maximum width in the range of 300 micrometers.

32. The implant according to at least one of claims 1-31, characterized in that the surface of said grooves comprises a metal layer produced by sputtering.

33. The implant according to claim 32, characterized in that said metal layer is a titanium layer produced by sputtering.

34. The implant according to claim 33, characterized in that said metal layer extends across the spaces between the grooves.

35. The implant according to claim 32 or 33, characterized in that said metal layer extends across the radial bands (17, 163, 192, 226) between the sections.

36. The implant according to claim 32 or 33, characterized in that said metal layer extends over the head area (12, 276).

37. A method of manufacturing a bone implant, especially a tooth implant, having an implant body (11) comprising a plurality of grooves (20) at its perimeter which extend along the longitudinal axis of the implant body or at an acute angle thereto, the dimensions of which correspond to the dimensions of the bone tissue osteons which attach to said groove-shaped recesses, and the surface of which is divided into a number of sections (16) along its longitudinal axis, characterized in that a blank of said implant body (11, 270) is produced in which grooves (20, 50, 70, 110, 231) are produced in at least one of said sections (16, 231) by a material removing process which form an angle α radial the longitudinal axis of said implant body (11, 270) and have varying depths across their lengths.

38. The method according to claim 37, characterized in that said grooves are produced by metal cutting using a synchronous feed of the implant body and the cutting tool.

39. The method according to claim 38, characterized in that said synchronous feed is selected such that wedge-shaped grooves (20) are produced at the implemented perimeter.

40. The method according to claim 38, characterized in that said synchronous feed is selected such that grooves (130) form a first angle α radial the longitudinal axis (41) of implant body (11) over a segment of one of sections (16) and form a second angle β radial the longitudinal axis (41) of said implant body (11) over another segment of the same section (16).

41. The method according to claim 40, characterized in that grooves (130) are of a form which approximates a diamond-shape profile at the implemented perimeter.

42. The method according to claim 41, characterized in that the virtually diamond-shaped grooves (140) extend in coiled fashion at the perimeter.

43. The method according to claim 38, characterized in that the synchronous feed changes from section to section.

44. The method according to claim 37, characterized in that grooves (271) in the head area of head section (272) extend to an edge region (276) which runs virtually parallel to the head boundary of the implant.

45. The method according to claim 37, characterized in that the synchronous feed is selected such that grooves (231) will exhibit peripheral curvature in at least one of sections (232).

46. The method according to claim 45, characterized in that the angle of curvature (σ) to grooves (231) is smaller at one of the edges of said section (232) than the angle of curvature (σ′) to grooves (231) at the other edge of said section (232).

47. The method according to claim 45, characterized in that the grooves (231) are narrow and shallow at one of the edges of said section (232) and exhibit their maximum width and depth at the other edge of said section (232).

48. The method according to claim 45, characterized in that the grooves (251) are narrow and shallow at both edges of said section (252) and exhibit their maximum width and depth therebetween (255).

49. The method according to claim 48, characterized in that the profile to grooves (251) at the implemented perimeter takes the form of elongated curved segments.

50. The method according to claim 48, characterized in that grooves (251) exhibit their maximum width and depth in an area (255) in close proximity to the center of their longitudinal extension.

51. The method according to claim 48, characterized in that grooves (251) exhibit their maximum width and depth in an area at one-third their longitudinal extension relative one of the two edges.

52. The method according to at least one of claims 37-51, characterized in that the additional step of sputtering a metal layer on the surface of the grooves is realized.

53. The method according to claim 52, characterized in that said metal layer is a titanium layer produced by sputtering.

54. The method according to claim 52 or 53, characterized in that said metal layer extends across the spaces between the grooves.

55. The method according to claim 52 or 53, characterized in that said metal layer extends across the radial bands (17, 163, 192, 226) between the sections.

56. The method according to claim 52 or 53, characterized in that said metal layer extends over the head area (12, 276).
